(19) United States
(12) Patent Application Publication
(10) Pub. No.: US 2005/0124958 A1
(43) Pub. Date: Jun. 9, 2005

(54) Disposable sanitary napkin with discrete flaps and method for manufacturing same

(76) Inventor: Henri Brisebois, Lachenaie (CA)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/726,717

(22) Filed: Dec. 3, 2003

(51) Int. Cl. B32B 31/00; A61F 13/15

(52) U.S. Cl. 604/385.04; 156/256; 156/290

(57) ABSTRACT

A disposable sanitary napkin with a main body and a pair of discrete flaps. Each flap includes an attachment portion secured to the main body. The attachment portion has a first end zone and a second end zone. Each end zone is co-extensive with a portion of a respective transversally extending side of the main body. The flap also includes a projecting portion that extends from the attachment portion. When the sanitary napkin is in use, the projecting portion projects laterally from a longitudinal side of the main body. The projecting portion has at least two layers. The two layers are bonded to one another and form a seal line extending along the periphery of the projecting portion.

### FIELD OF THE INVENTION

The present invention relates to disposable sanitary napkins. The sanitary napkin features at least one multi-layer flap that is discrete from the main body of the sanitary napkin. The present invention also relates to a method for manufacturing a sanitary napkin with at least one flap, and which generates a reduced amount of waste as compared to conventional manufacturing techniques.

### BACKGROUND OF THE INVENTION

Sanitary napkins are low cost mass produced articles. A typical manufacturing facility includes an assembly line where the various components of the sanitary napkin are progressively combined and laminated into a continuous web, which is cut transversely into discrete products.

The term “sanitary napkin”, as used herein, refers to an article that is worn by females in their undergarments adjacent to the pudendal region and which is intended to absorb and contain the various exudates that are discharged from the body (e.g., blood, menses, vaginal discharges and urine). Hence, the term “sanitary napkin” encompasses pantiliners as well as catamenial devices. The term “disposable” refers to articles that are intended to be discarded after a single use. That is, the articles are not intended to be laundered or otherwise restored or reused as an absorbent article.

To enhance the functionality of sanitary napkins, manufacturers are designing products with complex and sophisticated contour lines. For example, it is known to provide sanitary napkins with flaps having adhesive disposed thereon, the flaps being adapted to be folded over and secured to the undergarment of the user via the adhesive, thereby enabling the secure attachment of the sanitary napkin to the undergarment. With traditional manufacturing techniques, extensive trimming is required to produce the highly irregular contoured edge of such sanitary napkins, which generates a considerable amount of waste material. As a result, the manufacturing cost of the sanitary napkin increases because the starting material is used less efficiently.

Therefore, a clear need exists in the industry to develop sanitary napkins with one or more flaps, which can be produced with a reduced amount of waste material as compared to prior art sanitary napkins.

### SUMMARY OF THE INVENTION

In view of the forgoing, the present invention provides an improved disposable sanitary napkin which is intended to be worn in the undergarment of a user, the sanitary napkin being structured such that it can be manufactured with considerably less waste as compared to conventional napkins. The sanitary napkin according to the present invention includes a main body having a longitudinal axis; a transverse axis; first and second longitudinal sides extending along the longitudinal axis; and first and second transverse sides extending along the transverse axis. The sanitary napkin has at least one flap having at least two layers. The flap is discrete from the main body and includes an attachment portion extending along the longitudinal axis and secured to the main body. The attachment portion has a first end zone and a second end zone. The first end zone is co-extensive with at least a portion of the first transverse side and the second end zone is co-extensive with at least a portion of the second transverse side. The flap also includes a projecting portion that extends from the attachment portion. When the sanitary napkin is in use, the projecting portion projects laterally from the first longitudinal side of the main body. The two layers of the flap are bonded to one another and form a seal line extending along at least a portion of the periphery of the projecting portion.

The present invention also provides a method for manufacturing disposable sanitary napkins. The method comprises providing a continuous web formed from at least two superposed layers, the web having a longitudinal axis. The superposed layers are bonded to form an area of juncture that defines a cyclic pattern and extends along the longitudinal axis. The continuous web is severed at the area of juncture to form two longitudinally extending, continuous strips. Each of the strips have a plurality of projecting portions, each one of the projecting portions being arranged in spaced relationship to an adjacent projecting portion and each projecting portion being united to an adjacent projecting portion by a attachment portion. The method further includes providing a succession of absorbent systems, and advancing at least one of the strips and the succession of absorbent systems along paths which converge toward one another. The projecting portions of the strip are affixed to respective absorbent systems of the succession and the strip is transversely cut between adjacent absorbent systems to sever the attachment portions between the projecting portions.

### BRIEF DESCRIPTION OF THE DRAWINGS

A detailed description of examples of implementation of the present invention is provided herein below with reference to the following drawings, in which:

FIG. 1 is a bottom plan view of a sanitary napkin according to a first embodiment of the present invention;

FIG. 2 is a bottom plan view of the sanitary napkin of FIG. 1, the flaps of the sanitary napkin being shown in a position in which they project laterally from the longitudinal sides of the main body of the sanitary napkin;

FIG. 3a is a cross-sectional view of FIG. 2 taken along line 3-3 in FIG. 1;

FIG. 3b is a cross-sectional view of a sanitary napkin according to a second embodiment of the present invention;

FIG. 3c is a cross-sectional view of a sanitary napkin according to a third embodiment of the present invention;

FIG. 3d is a cross-sectional view of a sanitary napkin according to a fourth embodiment of the present invention;

FIG. 4 is a top plan view of a continuous multi-layer web from which flaps for the sanitary napkins according to the present invention are made, a portion of the multi-layer web being removed to show its structure;

FIG. 5a is a top plan view of the continuous web shown in FIG. 4 in which the layers of the web are bonded and form an area ofjuncture having a cyclic pattern;

FIG. 5b is a perspective view of an apparatus that may be used for obtaining the bonded continuous web of FIG. 5a;

FIG. 6 is a top plan view of the continuous web shown in FIG. 5a to which areas of adhesive material and their associated release papers have been added;

FIG. 7 is a top plan view of the continuous web shown in FIG. 6 that has been cut to form a pair of strips;

FIG. 8a is a top plan view of the continuous web of FIG. 7;

FIG. 8b is a top plan view of the continuous web of FIG. 7 according to another embodiment of the present invention;

FIG. 9 is a perspective view of an apparatus for putting the strips from the continuous web of FIG. 7 in phase;

FIG. 10 is a top plan view of the apparatus shown in FIG. 9;

FIG. 11 is a top plan view of the strips in phase as they leave the apparatus of FIG. 9;

FIG. 12 is a top plan view of a multi-layer continuous web, the web having a succession of absorbent systems to which the strips as shown in FIG. 11 are to be attached;

FIG. 13 is a perspective view of an apparatus for processing the web of FIG. 12 to seal the layers of the web to one another;

FIG. 14 is a perspective view of an apparatus for continuously joining of the web of FIG. 13 with the strips shown in FIG. 11;

FIG. 15 is a top plan view of the web produced by the apparatus in FIG. 14, the figure also showing the areas along which the web is cut to provide individual sanitary napkins;

FIG. 16 is a top plan view of the web of FIG. 15 when cut into individual sanitary napkins;

FIG. 17 is a perspective view of an apparatus for continuously joining of the web of FIG. 13 with two pairs of strips;

FIG. 18 is top plan view of a sanitary napkin as produced by the apparatus of FIG. 17, the sanitary napkin including partially overlapping flaps; and

FIG. 19 is a top plan view of a sanitary napkin as produced by the apparatus of FIG. 17, the sanitary napkin including flaps that are spaced from one another.

In the drawings, embodiments of the invention are illustrated by way of example. It is to be expressly understood that the description and drawings are only for purposes of illustration and as an aid to understanding, and are not intended to be a definition of the limits of the invention. Throughout the drawings, identical components are designated by the same reference numerals.

### DETAILED DESCRIPTION

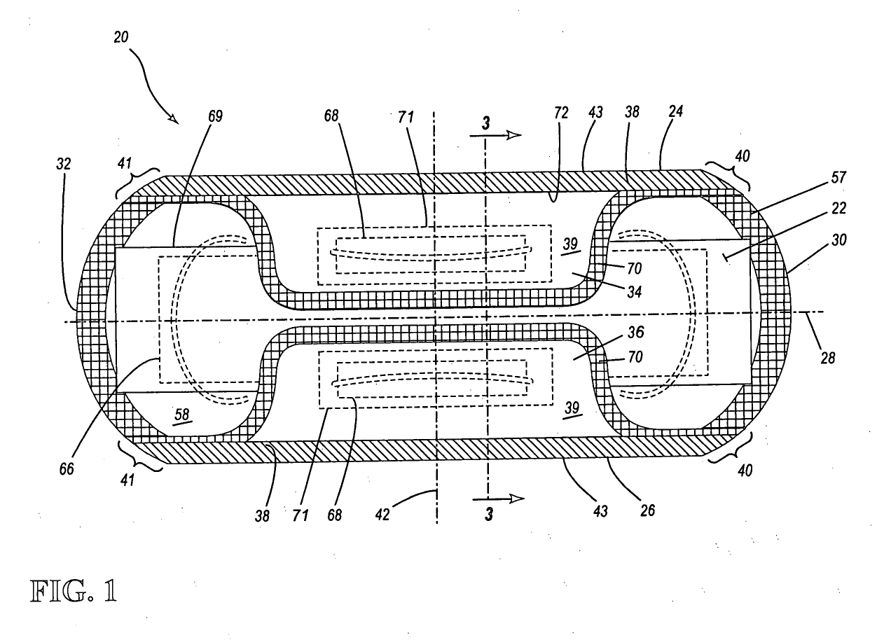

With reference to FIGS. 1, 2 and 3a there is shown a sanitary napkin 20 according to the present invention. The napkin 20 comprises a main body 22. The main body 22 has two mutually opposing longitudinally extending sides 24, 26, and an imaginary longitudinal centerline 28 running down the center of the napkin 20, generally equidistant from the longitudinally extending sides 24, 26.

The term “longitudinal”, as used herein, refers to a line, axis or direction in the plane of the sanitary napkin 20 that is generally aligned with (e.g., approximately parallel to) a vertical plane which bisects a standing wearer into left and right body halves when the sanitary napkin 20 is worn.

The main body 22 also has two mutually opposing transversely extending sides 30, 32. An imaginary transverse centerline 42 runs across the napkin 20, perpendicular to the longitudinal centerline 28.

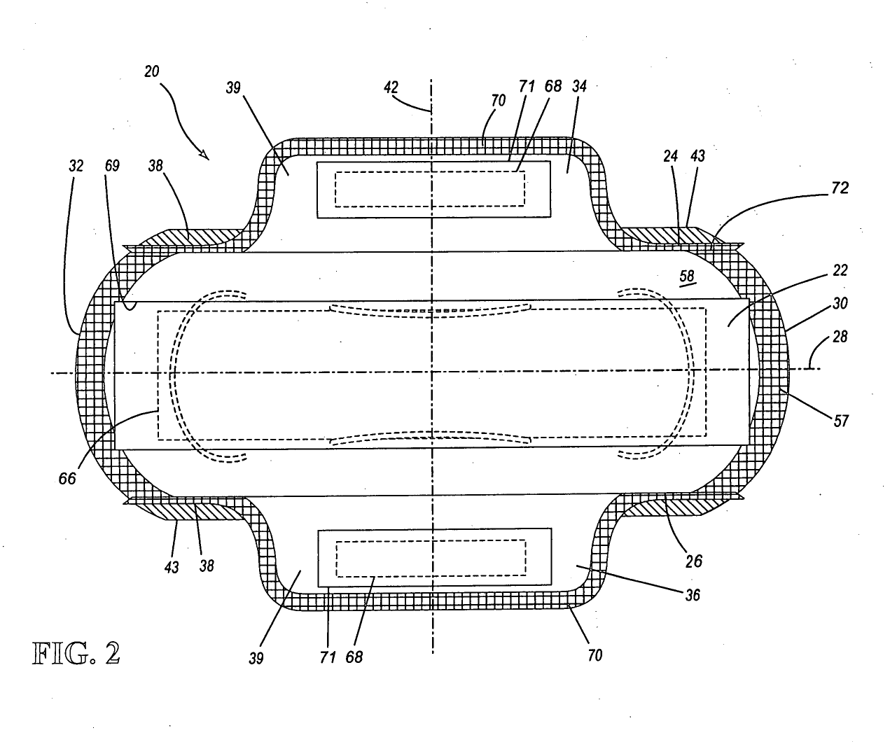

The sanitary napkin 20 has at least one flap, and in the embodiment shown in the Figures has two flaps 34, 36. As shown in FIG. 1, the sanitary napkin 20 is preferably manufactured such that the flaps 34, 36 are initially folded over the main body 22. Prior to use, as shown in FIG. 2, the flaps 34, 36 are unfolded such that they extend laterally outward from the main body 22. During use, the flaps 34, 36 are folded about the side edges of the undergarment in which the sanitary napkin 20 is installed.

The main body 22 has an upper surface that is adapted to be oriented toward the user and a lower surface that is adapted to face the undergarment of the wearer. The main body 22 may feature a single layer structure or a multilayer structure. In the case of a single layer structure, the upper and lower surfaces of the single layer define the upper and lower surfaces of the main body 22. In the case of a multilayer structure, different layers define the upper and lower surfaces of the main body 22.

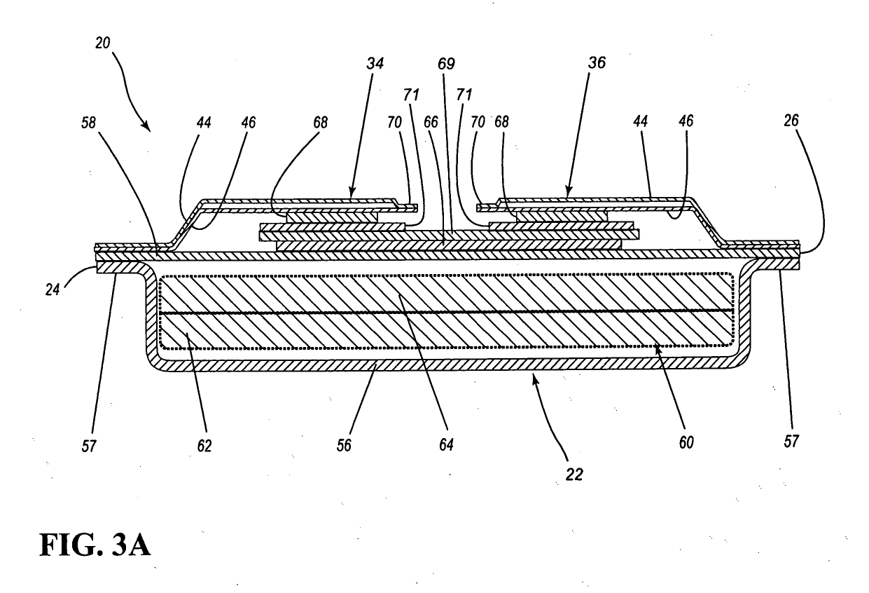

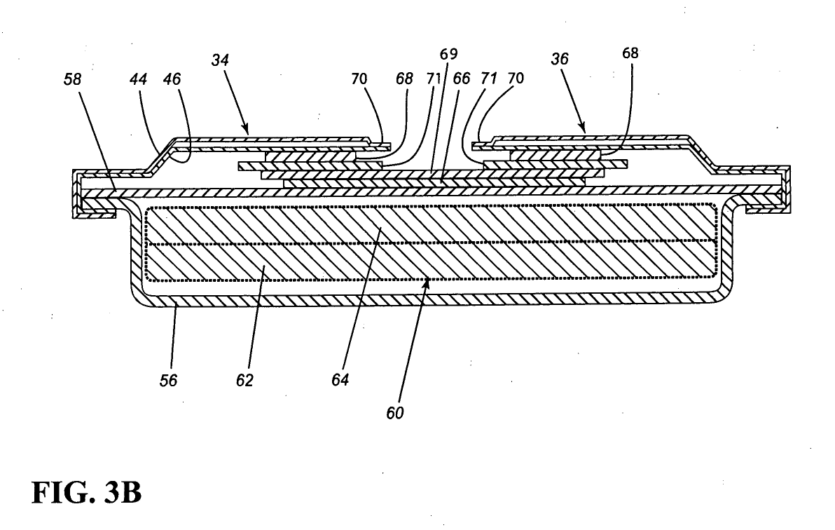

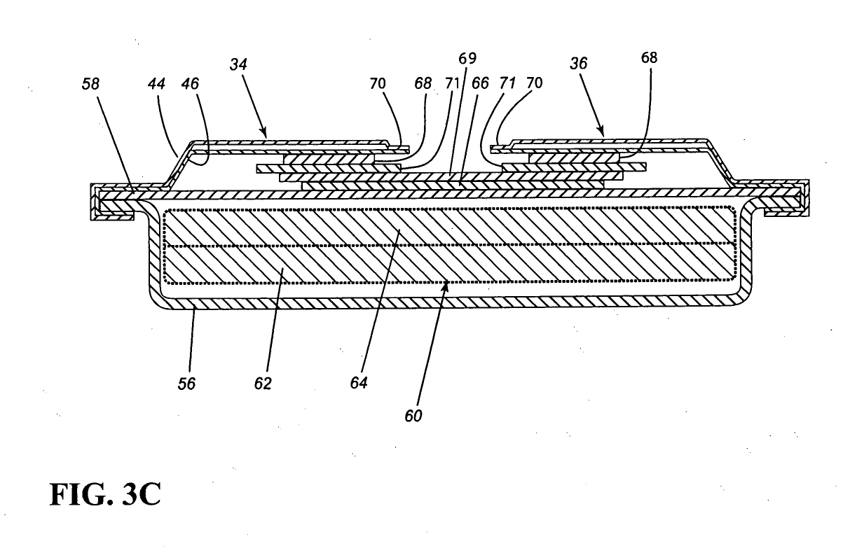

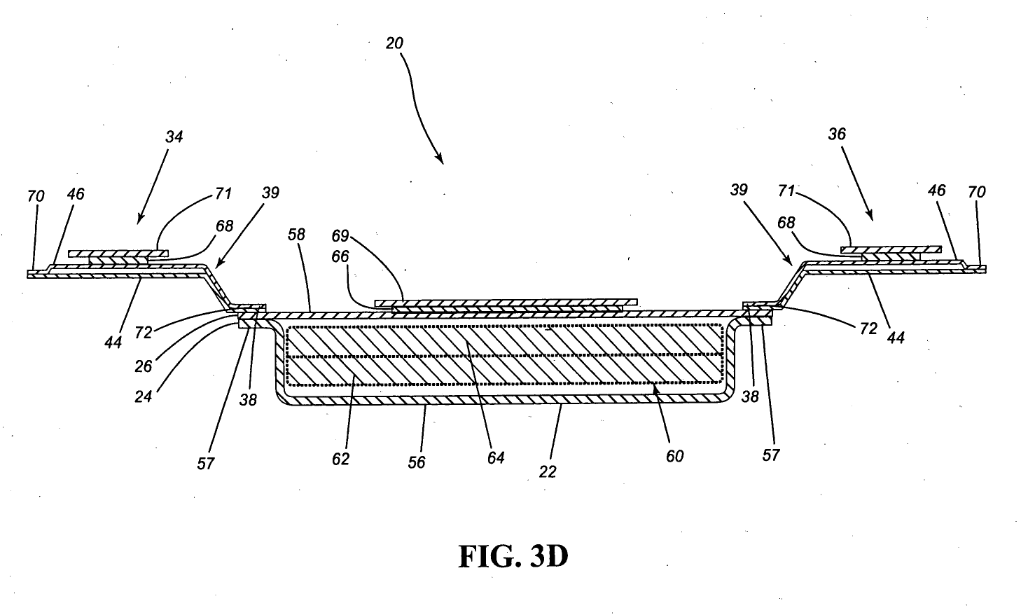

In accordance with a specific and non-limiting embodiment of the present invention as shown in FIG. 3a, the main body 22 is of a laminate construction and has at least three layers. More specifically, the main body 22 has a fluid-pervious cover layer 56, which is usually designated as the “cover layer”, that faces the body of a wearer when the napkin 20 is in use; a liquid-impervious barrier layer 58, which faces the wearer's undergarment when the napkin 20 is in use; and an absorbent system 60 therebetween. The absorbent system 60 has at least one absorbent component made of a single layer or multiple layers. The primary purpose of the absorbent system 60 is to absorb and retain body exudate. In the embodiment of the present invention depicted in FIG. 3a, the absorbent system 60 has two absorbent components, namely a first absorbent component 62 that comprises a single layer of material (commonly known as a “transfer layer”) and a second absorbent component 64 (commonly known as an “absorbent core”) that also comprises a single layer of material. The number of layers of material forming the first and the second absorbent components 62 and 64 is not essential since each absorbent component 62 and 64 may include more than one layer of material. It should be expressly noted that the first absorbent component 62 is optional and that the absorbent system 60 may include only the second absorbent component 64.

As shown in FIGS. 1, 2 and 3a, the fluid-pervious cover layer 56 and the liquid-impervious barrier layer 58 are sealed together along their peripheral edges to form a peripheral flange seal 57 containing the absorbent system 60. The peripheral flange seal 57 extends continuously around the absorbent system 60 to completely enclose the same. The peripheral flange seal 57 can be realized by using any means commonly known in the art for this purpose, such as by gluing, crimping, pressure and/or heat-sealing, ultrasonics, and the like.

Each of the components of the sanitary napkin 20 will be now described in further detail.

Main Body—Fluid—Pervious Cover Layer

The fluid-pervious cover layer 56 is the top layer of the sanitary napkin 20. The purpose of the fluid-pervious cover layer 56 is to provide an interface that would normally contact the body of the wearer when the sanitary napkin 20 is in use. The fluid-pervious cover layer 56 is porous to liquids since its main function is to capture as quickly as possible a discharge of bodily exudate and transfer it to the absorbent system 60 underneath.

The fluid-pervious cover layer 56 may be formed from an apertured thermoplastic film. Such films are common in the art. Because of the high porosity of such films, they accomplish the function of quickly transferring body exudate to the inner layers (i.e. the absorbent system 60) of the napkin 20.

The fluid-pervious cover layer 56 may alternatively be made of fibrous materials, such as non-woven fibrous materials. It may be composed of only one type of fiber, such as polyester, or it may alternatively be composed of bicomponent or conjugate fibers having a low melting point component and a high melting point component.

Bicomponent fibers may be made up of a polyester core and a polyethylene sheath. The use of appropriate bicomponent materials results in a fusible non-woven fabric. Using a fusible fabric increases the ease with which the fluid-pervious cover layer 56 may be mounted to the liquid-impervious barrier layer 58 at the peripheral flange seal 57.

The fibers may be selected from a variety of natural and synthetic materials such as nylon, polyester, rayon (in combination with other fibers), cotton acrylic fiber and the like and combinations thereof. It will be evident to the person skilled in the art that a wide variety of other types of non-woven fabric materials can also be used.

Main Body—Absorbent System—First Absorbent Component

Adjacent to the fluid-pervious cover layer 56 on its inner side and bonded thereto is an optional absorbent component 62 that may be used to form part of the absorbent system 60. The first absorbent component 62 provides the means of receiving body fluid from the fluid-pervious cover layer 56 and holding it until an underlying second absorbent component 64 has an opportunity to absorb the fluid.

The first absorbent component 62 may be made from one or more layers of material. In the embodiment of a sanitary napkin according to the present invention shown in the figures, the first absorbent component 62 is shown as a single layer, however as stated above, the absorbent component 62 may have a multi-layer structure if desired.

The first absorbent component 62 is preferably more dense and has a larger proportion of smaller pores than the fluid-pervious cover layer 56. These attributes allow the first absorbent component 62 to contain body fluid and hold it away from the inner side of the fluid-pervious cover layer 56, thereby preventing the fluid from re-wetting the fluid-pervious cover layer 56 and its surface. However, the first absorbent component 62 is preferably not so dense as to prevent the passage of the fluid into the underlying second absorbent component 64.

The first absorbent component 62 may be composed of fibrous materials, such as wood pulp, polyester, rayon, flexible foam, or the like, or combinations thereof. The first absorbent component 62 may also comprise thermoplastic fibers for the purpose of stabilizing the layer and maintaining its structural integrity. The first absorbent component 62 may be treated with surfactant on one or both sides in order to increase its wettability, although generally the first absorbent component 62 is relatively hydrophilic and may not require treatment. The first absorbent component 62 is preferably bonded on both sides to the adjacent layers, i.e. the fluid-pervious cover layer 56 and the underlying second absorbent component 64.

Main Body—Absorbent System—Second Absorbent Component

Immediately subjacent to and bonded to the first absorbent component 62 is the second absorbent component 64. As is the case with the first absorbent component 62, the second absorbent component 64 may also be made from one or more layers of material. FIGS. 1, 2 and 3a illustrate an embodiment of the sanitary napkin according to the present invention wherein the second absorbent component 64 consists of a single layer.

The second absorbent component 64 has a large liquid-holding capacity and is extremely retentive. The second absorbent component 64 may comprise pulp fluff material and may optionally include other absorbent materials or non-absorbent materials such as conjugate fibers, fusible fibers, binders, sphagnum moss, superabsorbents, and the like and combinations thereof.

In one embodiment, the second absorbent component 64 is a blend or mixture of cellulosic pulp fibers and superabsorbent disposed in and amongst fibers of the pulp. Preferably, the second absorbent component 64 contains from about 40 weight percent to about 95 weight percent cellulosic fibers, and from about 5 weight percent to about 60 weight percent SAP (superabsorbent polymers). By way of example, 10 weight percent SAP means 10 μm² SAP per 100 g/m² basis weight of the material.

Suitable cellulosic fibers that can be used in the second absorbent component 64 are well-known in the art and include wood pulp, cotton, flax and peat moss. Wood pulp is however preferred. Although both softwood and hardwood species can be used, softwood pulps are preferred. It is not necessary to treat cellulosic fibers with chemical debonding agents, cross-linking agents and the like.

Suitable SAP particles for use in the invention may be inorganic or organic crosslinked hydrophilic polymers, such as polyvinyl alcohols, polyethylene oxides, crosslinked starches, guar gum, xanthan gum, and the like and may be in the form of powder, grains, granules, or fibers.

Main Body—Liquid—Impervious Barrier Layer

Underlying the absorbent system 60 is a liquid-impervious barrier layer 58 which comprises liquid-impervious film material such as to prevent liquid that is entrapped in the absorbent system 60 from egressing the sanitary napkin 20 and staining the wearer's undergarment. The liquid-impervious barrier layer 58 can be made of polymeric film, such as polyethylene or a polyethylene/ethylvinyl acetate (EVA). The polymeric film is preferably capable of fully blocking the passage of liquid or gas that may emanate from the absorbent system 60. Alternatively, breathable films may be used that allow passage of gas while blocking liquid.

Flaps

Since both flaps 34 and 36 are substantially identical, only flap 34 will be described herein. Flap 34 is a discrete component, which implies that both it and the sanitary napkin's main body 22 are initially formed separately and then attached in a subsequent procedure. More specifically, during a finite time interval, the main body 22 and flaps 34 share no common boundary and are independent bodies. In the embodiment depicted in FIGS. 1, 2, and 3a, flap 34 is positioned under the main body 22. Note that since FIG. 1 is a bottom view of the sanitary napkin 20, flap 34 is depicted as being positioned over the liquid-impervious barrier layer 58.

As shown in FIGS. 1 and 2, the flap 34 has an attachment portion 38 that extends in a direction that is substantially parallel to the longitudinal centerline 28, and a projecting portion 39 that extends from the attachment portion 38. As shown in FIG. 1, the attachment portion 38 has two end zones, namely end zone 40 and end zone 41 that are generally opposite to one another. The end zones 40 and 41 are co-extensive with respective portions of the transversely extending sides 30, 32 of the periphery of the main body 22. The attachment portion 38 also has an outer edge 43 that extends from the end zone 40 to the end zone 41. In the specific case shown in FIGS. 1 and 2, the outer edge 43 of flap 34 is co-extensive with the longitudinal side 24 and in the case of flap 36, the outer edge 43 is co-extensive with the longitudinal side 26.

The projecting portion 39 is formed integrally with the attachment portion 38. As shown in FIGS. 1 and 2, the projecting portion 39 features a width, as measured along the imaginary transverse centerline 42, that is greater than that of the attachment portion 38.

As shown in FIG. 3a, the flap 34 includes at least two layers of material that form an outer layer 44 and an inner layer 46. The outer layer 44 contacts the skin of the wearer when in use and is advantageously made of material that is generally soft and non-irritating to the skin, such as a material identical to the material of the fluid-pervious cover layer 56. It can, however, also be made of any other type of suitable material. With respect to inner layer 46, the latter is preferably made of liquid-impervious material such as a material identical to the material of the liquid-impervious barrier layer 58. However, any other type of suitable material could be used to make inner layer 46. Additional layers of material can be included between the outer and inner layers 44, 46 without departing from the spirit of the invention.

As shown in FIGS. 1 and 2, the outer and inner layers 44, 46 are bonded to one another to form a first seal 70 that extends continuously along the periphery of the flap 34. The first seal 70 is realized by using any means commonly known in the art for this purpose, such as by gluing, crimping, pressure and/or heat-sealing, ultrasonics, and the like. The first seal that joins outer and inner layers 44, 46 will be described in greater detail herein below with reference to a later figure.

In the example shown in FIGS. 1, 2 and 3a, the first seal 70 extends continuously from the first end zone 40 to the second end zone 41 along the periphery of the flap 34. The first seal 70 is the primary means of bonding the outer and inner layers 44, 46 to one another. In the embodiment shown in FIGS. 1 and 2, the outer and inner layers 44, 46 are un-bonded inwardly of the first seal 70. In an alternative embodiment not shown, the outer and inner layers 44, 46 can be bonded to one another inwardly of the first seal 70, however, the degree of bonding inwardly of the first seal 70 is not as strong as the degree of bonding caused by the first seal 70.

As shown in FIGS. 1 and 2, flap 34 is attached to the main body 22 by a second seal 72 that is applied along the attachment portion 38 of the flap 34 and along the longitudinally extending side 24 of the main body 22, thereby securing the attachment portion 38 of the flap 34 to the main body 22 of the sanitary napkin 20. The second seal 72 is realized by using any means commonly known in the art for this purpose, such as by gluing, crimping, pressure and/or heat-sealing, ultrasonics, and the like. Although second seal 72 is preferably continuous from the first end zone 40 to the second end zone 41, it could also be realized by a succession of local bonding sites spaced from one another and extending along the outer edge 43 and the longitudinally extending side 24. In addition to attaching the flap 34 to the main body 22, the second seal 72 also unites the outer and inner layers 44, 46 to one another along the outer edge 43.

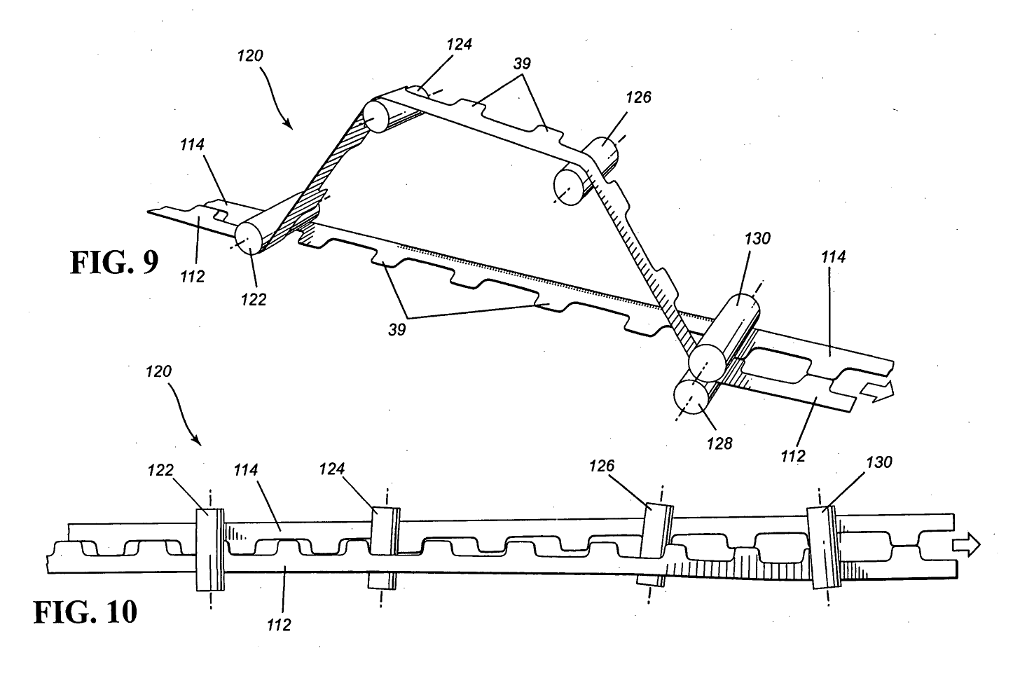

Although the above described embodiments of sanitary napkin 20 according to the present invention include discrete flaps that have been attached to the liquid-impervious barrier layer 58 of the sanitary napkin 20, it should be expressly understood that discrete flaps that are attached to the main body in other locations also remain within the scope of the invention. For example, in the embodiment of the invention depicted in FIG. 3b, the flaps 34 and 36 are secured to the fluid-pervious cover layer 56. As shown in FIG. 3c, the flaps 34 and 36 can also be secured to the main body in more than one location. Each flap 34, 36 in FIG. 3c, more precisely, is secured to both the fluid-pervious cover layer 56 and the liquid-impervious barrier layer 58. FIG. 3d shows another embodiment of the invention wherein flaps 34, 36 are attached to the main body 22 via their outer layers 44, such that when the flaps 34, 36 project outwardly, the projecting portion 39 of the flaps 34, 36 does not fold over the attachment portion 38. In the embodiment shown in FIG. 3D, when the flaps 34, 36 are positioned over the liquid-impervious layer 58, the projecting portion 39 of the flaps 34, 36 fold over the attachment portions 38. In order to obtain such a sanitary napkin, an apparatus similar to that which will be described in relation to FIGS. 9 and 10 can be used.

Adhesive Fasteners

Referring to FIGS. 3a, 3b, 3c and 3d, to secure the napkin 20 to the undergarment of a wearer, the liquid-impervious barrier layer 58 is provided with an adhesive fastener 66 comprising an area of standard adhesive material on the surface thereof that is intended to contact the wearer's undergarment. Additionally, there is an adhesive fastener 68 located on each of the flaps 34 and 36 that is intended to contact the underside of the wearer's undergarment. A single standard release paper 69 covers the adhesive fastener 66 positioned on the underside of the main body 22 while the adhesive fasteners 68 on the flaps 34, 36 are covered by respective release papers 71. The release papers 69 and 71 are of a conventional construction, such as silicone coated wet-laid Kraft wood pulp, for example.

Method of Manufacture

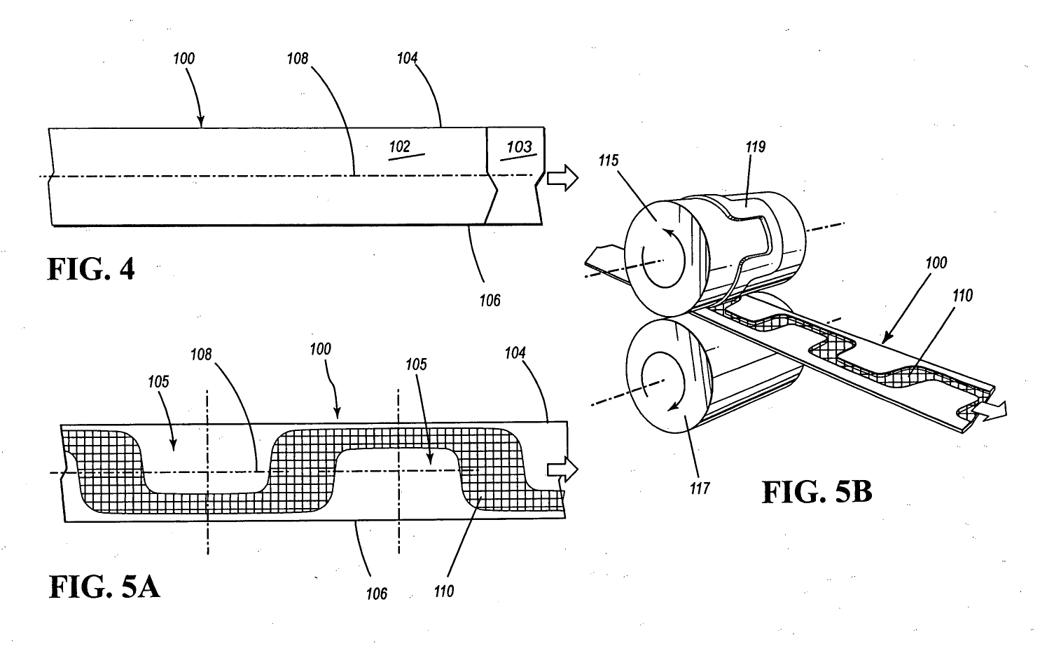

FIG. 4 shows a multi-layer web from which the flaps 34, 36 of sanitary napkin 20 are made. The web is designated by the reference numeral 100 and includes at least two layers. In a specific example of the invention depicted in the figures, the web 100 has an upper layer 102 that is preferably made of liquid-impervious material, such as the material used for making the liquid-impervious barrier layer 58. In FIG. 4 the web 100 has been depicted with a portion of the upper layer 102 removed to reveal an underlying lower layer 103 that is preferably made of a material that is soft and non-irritating to the skin, such as the material used for the manufacture of the fluid-pervious cover layer 56.

The web 100 has a continuous length and a constant width that is defined between side edges 104 and 106 that are parallel and extend along a longitudinal axis 108, which is parallel to the machine direction of the web 100. It should be noted that the web 100 is not restricted to only two layers, as more than two layers can be provided without departing from the spirit of the invention.

As shown in FIG. 5a, the two layers of web 100 are then bonded together at an area of juncture 110. The area of juncture 110 is constrained between the side edges 104 and 106 and has a cyclical pattern. Preferably, but not necessarily, the cyclical pattern defines a wave, such as a sinusoidal wave, for example, which oscillates evenly (i.e., of constant amplitude) around the longitudinal axis 108 of the web 100. The area of the web located outside of the area juncture 110, i.e. the portion that is not bonded during the bonding procedure, defines a plurality of alternating web portions 110 that extend transversely across from opposite sides of the web as shown. Each of the web portions 105 will ultimately form the flaps 34 and 36 of the napkin 20. The bonding of the upper and lower layers 102, 103 along the area of juncture 110 can be realized by using any means commonly known in the art for this purpose, such as by gluing, crimping, pressure and/or heat-sealing, ultrasonics, and the like. Although not shown in the figures, the bonding can also be realized by a succession of intermittent bonding sites spaced from one another.

When the area of juncture 110 is formed by heat-sealing, an apparatus of the type depicted in FIG. 5b may be used to perform the bonding step. The apparatus depicted in FIG. 5b includes a pair of rolls 115, 117 defining a nip between them. One of the rolls is a sealing roll 115 and the other is an anvil roll 117. One or both rolls is/are heated to a temperature consistent with the materials of the layers. The sealing roll 115 has on its periphery a circumferential projection 119 that contacts the web 100 as the latter passes through the nip. The projection 119 is designed such as to apply pressure on the area of the web 100 where the layers 102, 103 are to be bonded, thus forming the area of juncture 110.

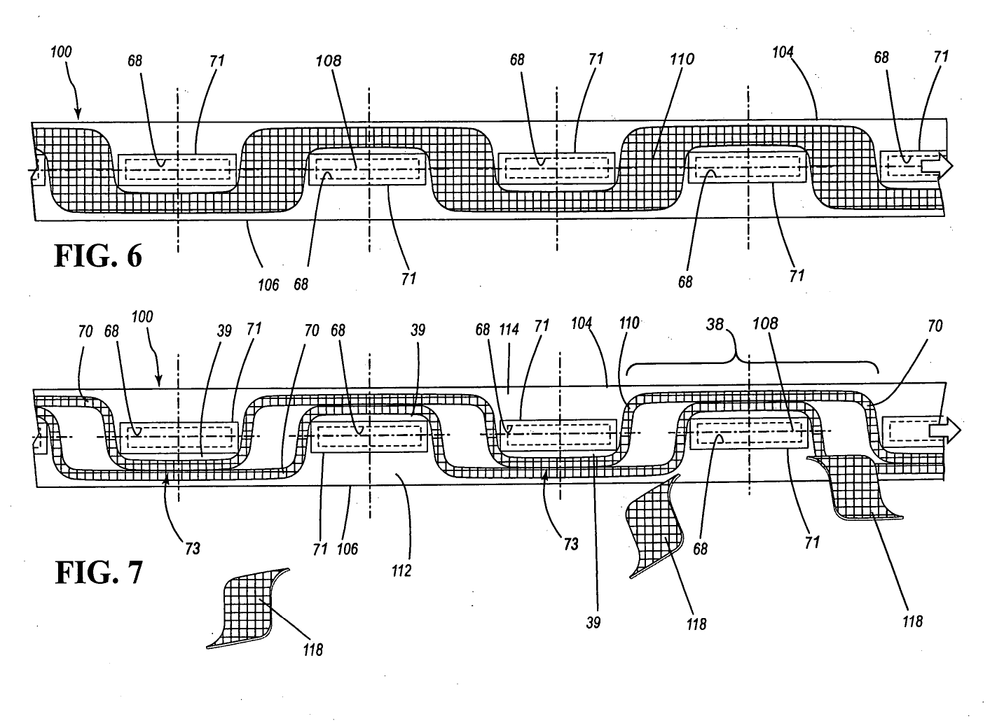

As shown in FIG. 6, after the layers 102 and 103 of the web 100 have been bonded to one another along the area of juncture 110 adhesive fasteners 68 are applied to the web 100. Specifically, adhesive fasteners 68 are applied to the web 100 on layer 102 and are then covered with release papers 71. Note that the adhesive fasteners 68 and their corresponding release papers 71 are applied in each transversely extending projection portion 39 delimited by the area of juncture 110. The application of the adhesive fasteners 68 and the release papers 71 can be performed with any suitable means known in the art.

FIG. 7 shows the web 100 having been severed lengthwise within the area of juncture 110 to form two longitudinally extending strips 112 and 114. In a first example of implementation, the two longitudinally extending strips 112 and 114 are severed via a first cutting operation, which cuts away sections 118 from within the area of juncture, and a second cutting operation, which forms a cut 73 along the area of juncture between the attachment portions 38 and the projecting portions 39. In a non-limiting example of implementation, these two cutting operations can be performed by stamping the web with stamps having blade patterns suitable for performing the two cutting operations described.

In a second example of implementation, the web 100 is severed by a cutting station that includes a pair of rolls that define a nip therebetween. One roll is a cutting roll and the other roll is an anvil roll. The cutting roll has on its periphery a pattern of blades for contacting the web 100 as the latter passes through the nip. The blade pattern is designed such as to apply a cutting force on the area of the web 100 that is desired to be cut. As such, in order to perform the cutting operation, the web 100 is fed through the nip such that the web 100 is cut along line 73, and along the lines surrounding sections 118. It should be understood that in such an embodiment, the two sides of section 118 are cut simultaneously. As such, the blade pattern on the cutting roll includes a single blade at the region for cutting along line 73, which splits into two blades for cutting the two lines around section 118. The two blades then re-combines into one blade for cutting the line 73 between the next attachment portion 38 and projecting portion 39.

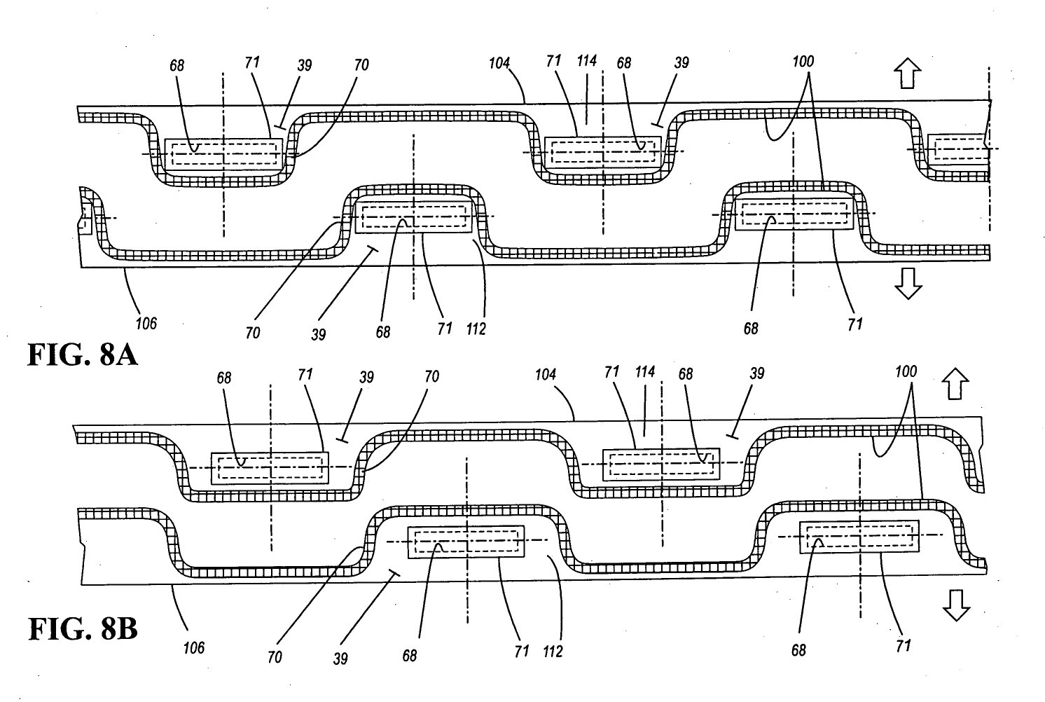

Shown in FIG. 8a are the resulting strips 112, 114, from FIG. 7, which have been separated in the direction of the arrows shown, once sections 118 have been removed. As shown, each strip 112, 114 includes a succession of the projecting portions 39 united to one another by the attachment portions 38. As indicated previously, the projecting portions 39 are characterized by a width, as measured in a direction that is perpendicular to the longitudinal axis 108. The width is generally greater than that of the attachment portions 38.

In the embodiment shown in FIG. 7, the sections 118 are cut from the area of juncture 110 and the remaining portions of the area of juncture 110 form the first seal 70 of the flaps 34 and 36. Although FIG. 7 depicts sections 118 as being within the area of juncture 110, meaning that they are bonded together, it should be specifically understood that the sections 118 could also not be bonded together, meaning that the first seal 70 would define the only area of juncture 110.

The size of sections 118 can be modified in order to alter the length of the projecting portions 39. For example, FIG. 8a depicts the strips 112 and 114 from FIG. 7, once the sections 118 have been removed and the strips have been separated, and FIG. 8b shows strips 112 and 114 formed from a web, wherein no sections 18 were cut out. As such, the projecting portions 39 shown in FIG. 8b have an increased dimension along the longitudinal axis 108 since the cutting operation performed on the web 100 of FIG. 8b is essentially a slit that separates the web 100 in two parts without the removal of any material.

As previously stated, the severing is done such that the resulting projecting portions 39 will be provided with the first seal 70. The severing can be effected with any suitable means. As described above, on example is the use of a cutting station, that includes a cutting roll and an anvil roll. The continuous web 100 passes through the nip formed by the cutting roll and the anvil roll. The cutting roll includes a peripheral cutting blade that severs the continuous web according to the selected pattern.

Although the above figures relate to a continuous web 100 that features upper and lower layers 102, 103 of identical width, it should be specifically understood that a continuous web 100 that is composed of upper and lower layers 102, 103 of differing widths remains within the scope of the present invention. Upper layer 102, for example, could be larger than lower layer 103 and, for example, positioned in the center of the latter. Such an arrangement could therefore result in strips 112, 114 where the projecting portions 39 are multi layers structures while the attachment portions 38 are composed of a single layer.

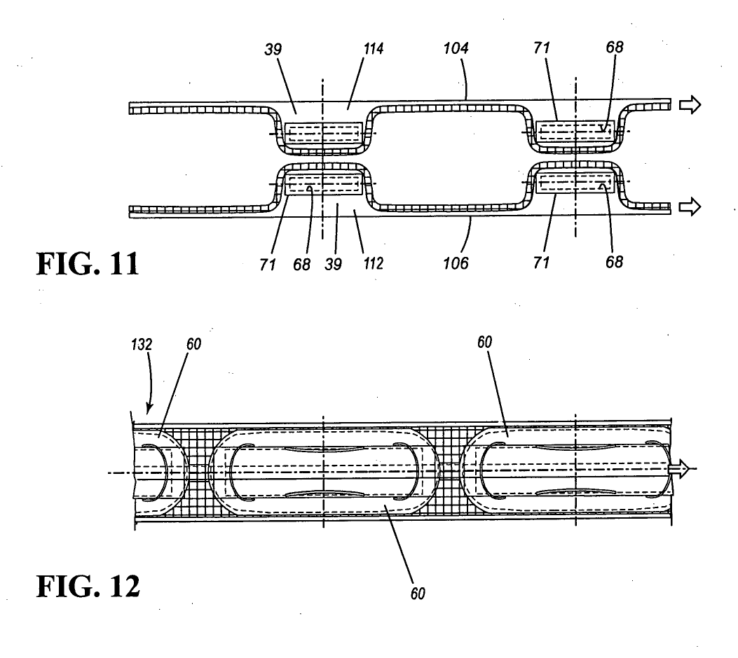

Once cut, the strips obtained from the continuous web 100 are processed by an apparatus 120, as shown in FIGS. 9 and 10, that puts them in phase with one another. The apparatus 120 comprises guide rollers 122, 124, 126, 128 and 130 that establish different paths of travel for the strips 112 and 114. The strip 112 follows a path of travel that is longer than the path followed by the strip 114 and, as a result, the strip 112 is delayed such as to bring the projecting portions 39 on the strip 112 in phase with the projecting portions 39 on the strip 114. The strips 112 and 114, when in phase, are shown at FIG. 11. Note that several features, such as the adhesive fasteners 68 and the release papers 71, are not shown in FIGS. 9 and 10 for the sake of clarity.

In accordance with an alternative embodiment, strips 112 and 114, in addition to being put in phase with respect to one another, can be crossed such that the projection portions 39 of each strip do not face one another but rather face outwardly with respect to one another. This would be the case when manufacturing a napkin 20 as shown in FIG. 3d. Alternatively, the strips 112 and 114 could be crossed and placed in a partially overlapping relationship such that the attachment portions 38 of each strip overlap each other.

The method according to the invention also includes providing a plurality of absorbent systems. FIG. 12 shows a sequence of absorbent systems 60. The absorbent systems 60, which can comprise a single layer of material or multiple layers of material, are carried in a continuous web 132. The continuous web 132 generally includes three superposed elements. The first element is a bottom layer that is continuous and is made from the material of the liquid-impervious barrier layer 58. The second element is the series of absorbent systems 60 that are placed on top of the bottom layer in a spaced apart relationship. The spacing between the absorbent systems 60 is generally selected to match the spacing between the projecting portions 39 on the strips 112 and 114. The third element is a top layer made from the material of the fluid-pervious cover layer 56.

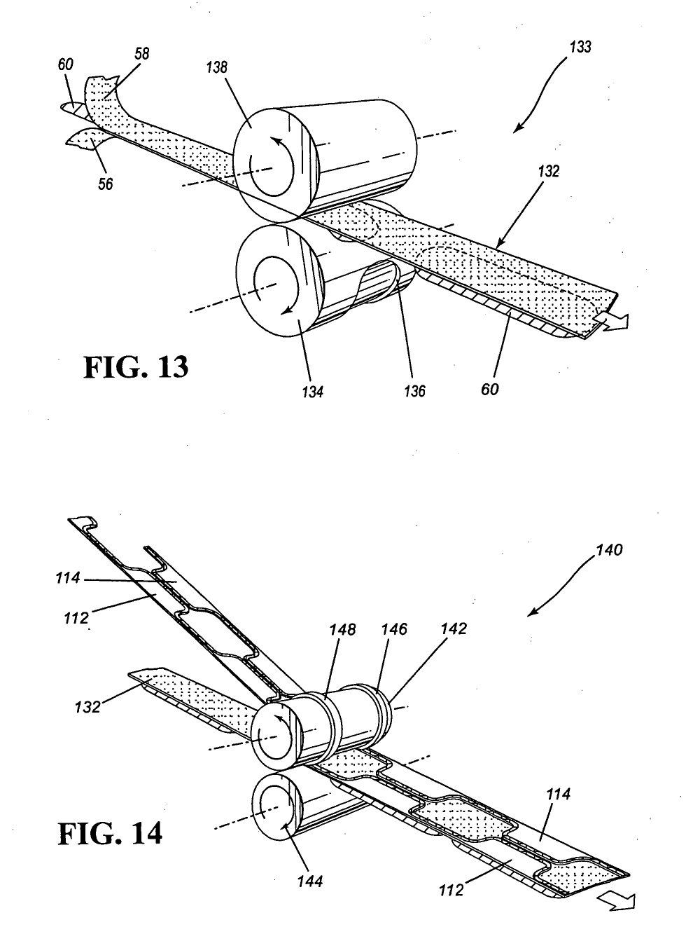

The bottom layer and the top layer of the web 132 are then bonded to one another at the areas surrounding the absorbent systems 60. In FIG. 12, these regions of bonding 61 are shown by the cross-hatchings. An apparatus that may be used to bond the bottom layer to the top layer is shown in FIG. 13. The apparatus 133 includes a heated sealing roll 134 featuring a cavity 136 that is dimensioned to receive an absorbent system 60. The apparatus 133 also includes an anvil roll 138, which defines a nip with the sealing roll 134. The web 132 passes through the nip and is synchronized with the roll 134 such that the absorbent systems 60 mate with the cavity 136. Accordingly, little pressure and heat is applied to the absorbent systems 60, while the remainder of the web 132 is subjected to sufficient levels of pressure and heat to heat-seal the bottom layer and the top layer of the web 132 all around the absorbent systems 60. The bonding of the bottom layer and the top layer of the web 132 can also be done through other methods, such as crimping, adhesives and ultrasonics, among others.

The web 132 as it leaves the apparatus 133 can be viewed as a succession of absorbent systems 60, where the top layer of the web 132 forms the liquid-impervious barrier layer 58 and the bottom layer forms the fluid-pervious cover layer 56 of each absorbent system 60.

FIG. 14 shows an apparatus 140 to unite, in a continuous fashion, the web 132 with the strips 112 and 114. The apparatus 140 includes a heat-sealing station with a heated sealing roll 142 and an anvil roll 144 which together define a nip through which are directed the strips 112 and 114 and the web 132. As the strips 112, 114, and the web 132 pass through the nip, they converge toward one another and are bonded to one another. In particular, the bonding is made by two peripherally extending projections 146, 148 formed on the roll 142.

After the heat-sealing station, the strips 112, 114 that have been united with web 132 in such a manner that the projecting portions 39 of each strip 112, 114 are centered with respect to the transverse centerlines of the absorbent systems 60. However, it should be expressly understood that uniting the strips 112, 114 with web 132 such that the projecting portions 39 are offset with respect to the transverse centerline of each successive absorbent system 60 remains within the scope of the invention.

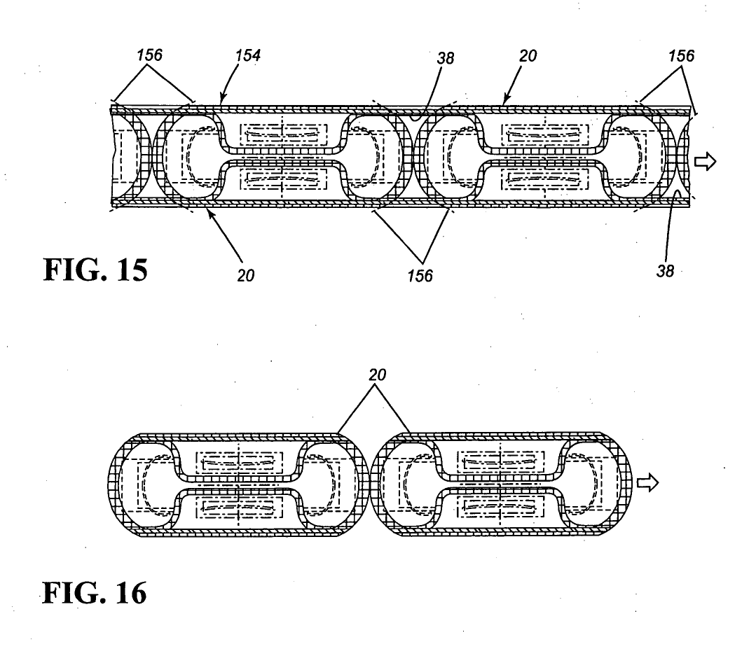

The web 154 that results from the combination of the web 132 and the strips 112 and 114 is shown in FIG. 15. The web 154 is then cut transversely at the areas 156, identified by the curved dashed lines, to provide discrete sanitary napkins 20. The cutting operation thus severs the regions of the attachment portions 38 located between napkins 20.

The cutting can be done with any suitable means. A possible example is a cutting station including a cutting roll and an anvil roll through which the web 154 passes. The cutting roll has on its circumference cutting blades that perform the cutting of the web 154 as the cutting roll turns. The shape of the cutting blades is such that as the rolls turn, they sever the web 154 only at the areas 156.

FIG. 16 shows a succession of sanitary napkins 20 once the cutting operation has been completed and the waste material removed.

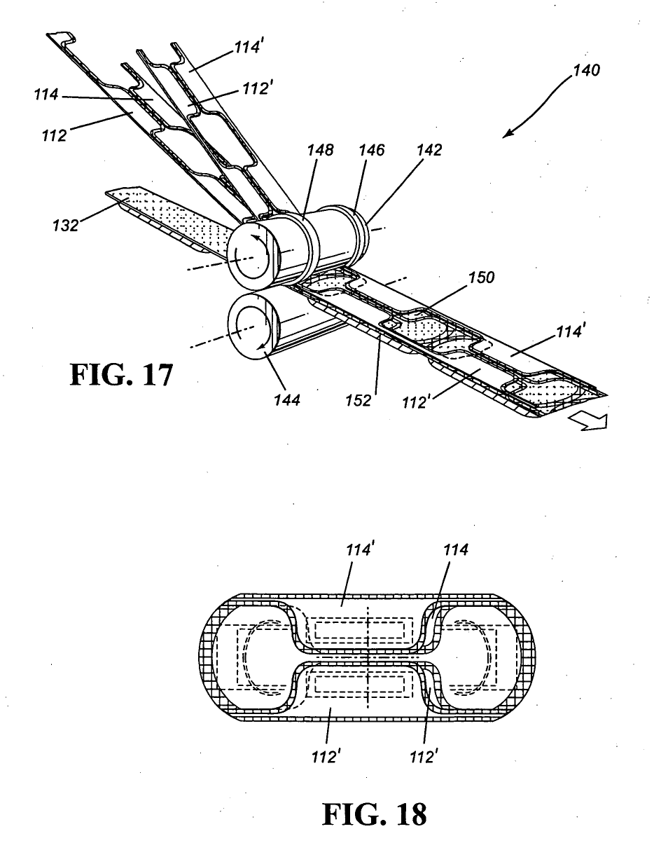

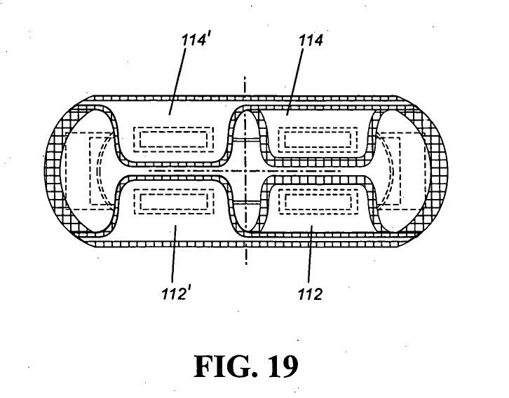

Although the above method describes a single pair of flaps 34, 36 attached to an individual sanitary napkin, it should be specifically understood that attaching more than one pair of flaps 34, 36 to a single sanitary napkin remains within the scope of this invention. In order to do so, an apparatus similar to that shown in FIG. 14 could be used to unite the web 132 with first and second pairs of strips 112, 114 and 112′, 114′. This is shown in FIG. 17. Alternatively, two apparatuses similar to that shown in FIGS. 14 and 17 could be placed in series. As a result, the first apparatus could unite the web 132 with the first pair of strips 112, 114 and the second apparatus could be used to attach the second pair of strips 112′, 114′. An example of the product resulting from such a method is shown in simplified manner in FIG. 18. As depicted, each side of the sanitary napkin features a pair of flaps that partially overlap one another. It should be expressly understood, however, that flaps which are distanced from one another and therefore don't overlap, as shown in FIG. 19, also remain within the scope of the present invention.

Although various embodiments have been illustrated, this was for the purpose of describing, but not limiting, the invention. Various modifications will become apparent to those skilled in the art and are within the scope of this invention, which is defined more particularly by the attached claims.

1. A method for manufacturing disposable sanitary napkins intended to be worn in undergarments of users, each sanitary napkin having a main body that comprises an absorbent system, the main body including a pair of mutually opposite sides and at least one flap projecting from one of said mutually opposite sides, said method comprising:
a) providing a continuous web having a longitudinal axis and at least two superposed layers;
b) bonding said superposed layers to form an area of juncture on said continuous web, said area of juncture defining a cyclic pattern and extending along said longitudinal axis;
c) severing said continuous web within said area of juncture to form two strips, each strip having:
i) a plurality of projecting portions in a spaced apart relationship;
ii) at least one attachment portion between each of said projecting portions that unite said projecting portions to one another;
d) providing a succession of absorbent systems;
e) advancing at least one of said strips and said succession of absorbent systems along respective paths that converge toward one another;
f) securing respective projecting portions of said one strip to respective absorbent systems of said succession;
g) cutting transversally said at least one strip between absorbent systems of said succession to sever the attachment portions between the projecting portions of said one strip.

2. A method as defined in claim 1, wherein said main body is a multi-layer structure.

3. A method as described in claim 2, wherein said main body includes a fluid-pervious layer overlying said absorbent system, said fluid-pervious layer being oriented toward the user when the disposable sanitary napkin is in use.

4. A method as defined in claim 3, wherein said main body further includes a liquid-impervious layer underlying said absorbent system, said liquid-impervious layer being oriented toward the undergarment of the user when the disposable sanitary napkin is in use.

5. A method as defined in claim 3, wherein said projection portions of said at least one strip are secured to said fluid-pervious layer.

6. A method as defined in claim 4, wherein said projection portions of said at least one strip are secured to said liquid-impervious layer.

7. A method as defined in claim 4, wherein said projecting portions of said one strip are secured to said fluid-pervious layer and said liquid-impervious layer.

8. A method as defined in claim 1, further comprising applying adhesive fasteners before the severing of said continuous web.

9. A method as defined in claim 1, wherein said projecting portions of said two strips are secured to respective absorbent systems of said succession.

10. A method as defined in claim 1, wherein said plurality of projection portions are transversely projecting.

11. A method as defined in claim 1, wherein the securing is performed such that one absorbent body is secured in association a projecting portion from each of said strips formed from said continuous web.

12. A method as defined in claim 1, wherein said two strips are first and second strips, said method further comprising:
providing a third strip, said third strip having a plurality of projecting portions in a spaced apart relationship and at least one attachment portion between each of said projecting portions that unite said projecting portions to one another; and
securing the projecting portions of said third strip to respective absorbent systems of said succession before the cutting such that the projecting portions of said third strip at least partially overlap the projecting portions of either one of said first and second strips.

13. A method as defined in claim 1, wherein said two strips are first and second strips, said method further comprising:
providing a third strip, said third strip having a plurality of projecting portions in a spaced apart relationship and at least one attachment portion between each of said projecting portions that unite said projecting portions to one another;
securing the projecting portions of said third strip to respective absorbent systems of said succession before the cutting such that the projecting portions of said third strip are spaced apart from the projecting portions of either one of said first and second strips.

14. A method as defined in claim 1, wherein the layers of said continuous web are of substantially identical widths.

15. A method as defined in claim 1, wherein the layers of said continuous web are of substantially differing widths.

16. A disposable sanitary napkin intended to be worn in the undergarment of a user, said sanitary napkin comprising;
a) a main body having:
i) a longitudinal axis;
ii) a transverse axis;
iii) first and second longitudinal sides extending along said longitudinal axis;
iv) first and second transverse sides extending along said transverse axis;
b) at least one flap being discrete from said main body and including:
i) an attachment portion secured to said main body, said attachment portion extending along said longitudinal axis, said attachment portion including a first end zone and a second end zone, said first end zone being coextensive with at least a portion of said first transverse side, said second end zone being coextensive with at least a portion of said second transverse side;
ii) a projecting portion that extends from said attachment portion, in use said projecting portion projects laterally from said first longitudinal side, said projecting portion having a periphery;
iii) said attachment portion and said projection portion having at least two layers, said at least two layers being bonded to one another to form a first seal that extends along at least a portion of the periphery of said projecting portion.

17. A disposable sanitary napkin as defined in claim 16, wherein said main body is a multi-layer structure.

18. A disposable sanitary napkin as defined in claim 17, wherein said main body includes a fluid-pervious layer that is oriented toward the user when said disposable sanitary napkin is in use.

19. A disposable sanitary napkin as defined in claim 18, wherein said main body further includes an absorbent system underlying said fluid-pervious layer.

20. A disposable sanitary napkin as defined in claim 19, wherein said absorbent system is a multi-layer structure.

21. A disposable sanitary napkin as defined in claim 19, wherein said main body further includes a liquid-impervious layer underlying said absorbent system, said liquid-impervious layer being oriented toward the undergarment of the user when said disposable sanitary napkin is in use.

22. A disposable sanitary napkin as defined in claim 21, wherein said attachment portion is secured to said liquid-impervious layer.

23. A disposable sanitary napkin as defined in claim 18, wherein said attachment portion is secured to said fluid-pervious layer.

24. A disposable sanitary napkin as defined in claim 21, wherein said attachment portion is secured to said fluid-pervious layer and said liquid-impervious layer.

25. A disposable sanitary napkin as defined in claim 16, wherein said at least one flap is a first flap, said sanitary napkin further comprising:
a) a second flap, said second flap being discrete from main body and including:
i) an attachment portion secured to said main body, the attachment portion of said second flap extending along said longitudinal axis, the attachment portion of said second flap including a first end zone and a second end zone, the first end zone of said second flap being coextensive with at least a portion of said first transverse side, the second end zone of said second flap being coextensive with at least a portion of said second transverse side;
ii) a projecting portion that extends from the attachment portion of said second flap, in use the projecting portion of said second flap projecting from said first longitudinal side.

26. A disposable sanitary napkin as defined in claim 25, wherein said projecting portion of said second flap and said projection portion of said first flap partially overlap one another.

25. A disposable sanitary napkin as defined in claim 25, wherein said projecting portion of said second flap and said projecting portion of said first flap are spaced from one another.
